(19) United States
(12) Patent Application Publication
(10) Pub. No.: US 2022/0143428 A1
(43) Pub. Date: May 12, 2022

(54) ACOUSTIC ENERGY TREATMENT

(71) Applicant: THE UNIVERSITY OF QUEENSLAND, St. Lucia, Queensland (AU)

(72) Inventors: Juergen GOETZ, St Lucia, Queensland (AU); Fabrice TURPIN, St Lucia, Queensland (AU); Daniel BLACKMORE, St Lucia, Queensland (AU)

(73) Assignee: THE UNIVERSITY OF QUEENSLAND, St. Lucia, Queensland (AU)

(21) Appl. No.: 17/433,949

(22) PCT Filed: Feb. 27, 2020
(86) PCT No.: PCT/AU2020/050173

§ 371 Date: Aug. 25, 2021

(30) Foreign Application Priority Data
Feb. 27, 2019 (AU) 2019900632

(51) Int. Cl. A61N 7/00; G16H 20/70

(52) U.S. Cl. CPC A61N 7/00; G16H 20/70; A61N 2007/0082; A61N 2007/0039; A61N 2007/0026

(57) ABSTRACT

The present invention relates to methods of restoring cognitive function in aged individuals that do not exhibit a clinically detectable neurodegenerative disease. In one aspect, the present invention provides a method of improving cognitive function in an aged individual that does not have a neurodegenerative disease characterized by aggregation of a pathological protein, the method comprising or consisting of: applying a clinically safe level of acoustic energy to sites within a region of the brain, thereby saturating or substantially saturating the region with acoustic energy, thereby improving cognitive function in the aged individual.

### CROSS-REFERENCE TO EARLIER APPLICATION

This application claims priority to Australian provisional application AU 2019900632, the entire contents of which is herein incorporated by reference in its entirety.

### FIELD OF THE INVENTION

The present invention relates to methods and apparatus for use in restoring and/or improving cognitive function in aged individuals that do not exhibit a clinically detectable neurodegenerative disease, preferably the cognitive function is memory or learning.

### BACKGROUND OF THE INVENTION

In the process of ageing, the human brain undergoes physiological and pathological forms of ageing. These forms of ageing differ. The latter is e.g. observed in Alzheimer's disease (AD) that is characterised by the deposition of amyloid as plaques and tau as tangles, in addition to neuronal loss. Physiological ageing frequently results in some degree of cognitive impairment, including decline in cognitive function that progresses with age and age-related changes in brain morphology and cerebrovascular function. Cognitive decline has been consistently reported with ageing across a range of cognitive domains including processing speed, attention, episodic memory, spatial ability and executive function. Brain imaging studies have revealed that these normal age-related cognitive declines are associated with decreases in both grey and white matter volume in the brain, with the fronto-striatal system most heavily compromised with ageing. These decreases in cortical volume can be attributed to a number of detrimental cellular processes involved with normal ageing. In addition to direct cellular damage, the brain is also indirectly impaired by insults to micro-vascular structures.

In the nervous system, aging is accompanied by structural and neurophysiological changes that drive cognitive decline. Included in these changes are synapse loss and the loss of neuronal function that results. Thus, although significant neuronal death is typically not observed during the natural aging process, neurons in the aging brain are vulnerable to sub-lethal age-related alterations in structure, synaptic integrity, and molecular processing at the synapse, all of which impair cognitive function. Accumulating evidence suggests that there is a neurogenetic component to cognitive impairment. For example, changes in the mammalian brain appear to parallel alterations in distinct learning and memory processes. In particular, alterations in the hippocampal formation are among the most prominent and consistent features observed in age-related cognitive impairment.

Therefore, there is a need for methods of reducing or preventing cognitive decline and/or improving or restoring cognitive function in aged individuals.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a method of improving or restoring cognitive function in an aged individual that does not have a neurodegenerative disease characterized by the presence of a pathological protein, the method comprising or consisting of:
applying a clinically safe level of acoustic energy to sites within a region of the brain, thereby saturating or substantially saturating the region with acoustic energy,
thereby improving or restoring cognitive function in the aged individual.

In one aspect, the present invention provides a method of inducing long-term potentiation in an aged individual that does not have a neurodegenerative disease characterized by the presence of a pathological protein, the method comprising or consisting of:
applying a clinically safe level of acoustic energy to sites within a region of the brain, thereby saturating or substantially saturating the region with acoustic energy, thereby inducing long-term potentiation in the aged individual.

In any aspect of the present invention, the aged individual may have cognitive impairment, Mild Cognitive Impairment, or delirium.

In another aspect, the present invention provides a method of improving Mild Cognitive Impairment (MCI) in an individual, the method comprising or consisting of:
applying a clinically safe level of acoustic energy to sites within a region of the brain, thereby saturating or substantially saturating the region with acoustic energy,
thereby improving Mild Cognitive Impairment (MCI) in the individual.

In any aspect of the present invention, the aged individual is at least 40, 45, 50, 55, 60, 65, 70, 75, 80, 85, 90 or 95 years old.

Preferably, the aged individual is at least 50, 55, 60, 65, 70, 75, 80, 85, 90 or 95 years old. Even more preferably, the aged individual is at least 60, 65, 70, 75, 80, 85, 90 or 95 years old.

In any aspect of the present invention, the aged individual does not have any biochemically and/or clinically detectable neurodegenerative disease characterized by the presence of a pathological protein. The pathological protein may be located extracellularly and/or intracellularly. The pathological protein may be in one or more of the following forms: oligomers, aggregates and/or deposits Preferably, the aged individual does not have any biochemically and/or clinically detectable neurodegenerative disease. Biochemical and clinical methods to detect neurodegenerative disease are known to the skilled person and include those described herein.

In one aspect, the present invention provides a method of improving memory in an aged individual that does not have a neurodegenerative disease characterized by presence of a pathological protein, the method comprising or consisting of:
applying a clinically safe level of acoustic energy to the region, thereby saturating or substantially saturating the region with acoustic energy;
thereby improving memory in the aged individual. The type of memory may be any one described herein, for example working memory, spatial memory, short-term memory, long-term memory, anterograde memory, retrograde memory, or memory retrieval.

In another aspect, the present invention provides a method of increasing neurogenesis in an aged individual that does not have a neurodegenerative disease characterized by presence of a pathological protein, the method comprising or consisting of:
applying a clinically safe level of acoustic energy to the region, thereby saturating or substantially saturating the region with acoustic energy;
thereby increasing neurogenesis in the aged individual.

In another aspect, the present invention provides use of a clinically safe level of acoustic energy for:
improving or restoring cognitive function in an aged individual that does not have a neurodegenerative disease characterized by the presence of a pathological protein;
inducing long-term potentiation in an aged individual that does not have a neurodegenerative disease characterized by the presence of a pathological protein;
improving Mild Cognitive Impairment (MCI) in an individual;
improving memory in an aged individual that does not have a neurodegenerative disease characterized by the presence of a pathological protein;
increasing neurogenesis in an aged individual that does not have a neurodegenerative disease characterized by presence of a pathological protein and/or
improving learning in an aged individual that does not have a neurodegenerative disease characterized by presence of a pathological protein
wherein the acoustic energy is applied to sites within a region of the brain, thereby saturating or substantially saturating the region with acoustic energy.

In another aspect, the present invention provides a clinically safe level of acoustic energy for use in:
improving or restoring cognitive function in an aged individual that does not have a neurodegenerative disease characterized by the presence of a pathological protein;
inducing long-term potentiation in an aged individual that does not have a neurodegenerative disease characterized by the presence of a pathological protein;
improving Mild Cognitive Impairment (MCI) in an individual;
improving memory in an aged individual that does not have a neurodegenerative disease characterized by the presence of a pathological protein;
increasing neurogenesis in an aged individual that does not have a neurodegenerative disease characterized by presence of a pathological protein and/or
improving learning in an aged individual that does not have a neurodegenerative disease characterized by presence of a pathological protein,
wherein the acoustic energy is applied to sites within a region of the brain, thereby saturating or substantially saturating the region with acoustic energy.

In another aspect, the present invention provides a method of improving memory in an aged individual with impaired memory function, the method comprising or consisting of:
providing an aged individual with impaired memory function;
identifying a region of the brain of the individual to be treated with acoustic energy;
applying a clinically safe level of acoustic energy to the region, thereby saturating or substantially saturating the region with acoustic energy;
wherein the aged individual does not have a neurodegenerative disease characterized by presence of a pathological protein;
thereby improving memory in the aged individual.

In another aspect, the present invention provides a method of improving cognitive function in an aged individual with impaired cognitive function, the method comprising or consisting of:
providing an individual with impaired cognitive function;
identifying a region of the brain of the individual to be treated with acoustic energy;
applying a clinically safe level of acoustic energy to the region, thereby saturating or substantially saturating the region with acoustic energy;
wherein the aged individual does not have a neurodegenerative disease characterized by presence of a pathological protein
thereby improving cognitive function in the individual.

In another aspect, the present invention provides use of a clinically safe level of acoustic energy for:
improving memory in an aged individual with impaired memory function, wherein the aged individual does not have a neurodegenerative disease characterized by presence of a pathological protein; and/or
improving cognitive function in an aged individual with impaired cognitive function, wherein the aged individual does not have a neurodegenerative disease characterized by presence of a pathological protein;
wherein the use comprises or consists of:
providing an aged individual with impaired cognitive function or impaired memory function;
identifying a region of the brain of the individual to be treated with the acoustic energy; and
applying a clinically safe level of the acoustic energy to the region, thereby saturating or substantially saturating the region with acoustic energy.

In another aspect, the present invention provides a clinically safe level of acoustic energy for use in:
improving memory in an aged individual with impaired memory function, wherein the aged individual does not have a neurodegenerative disease characterized by presence of a pathological protein; and/or
improving cognitive function in an aged individual with impaired cognitive function, wherein the aged individual does not have a neurodegenerative disease characterized by presence of a pathological protein;
wherein the use comprises or consists of:
providing an aged individual with impaired cognitive function or impaired memory function;
identifying a region of the brain of the individual to be treated with the acoustic energy; and
applying a clinically safe level of the acoustic energy to the region, thereby saturating or substantially saturating the region with acoustic energy.

In another aspect, the present invention provides a method of improving learning in an aged individual that does not have a neurodegenerative disease characterized by presence of a pathological protein, the method comprising or consisting of:
applying a clinically safe level of acoustic energy to the region, thereby saturating or substantially saturating the region with acoustic energy;
thereby improving learning in the aged individual. Specifically, in this aspect, the ability or capacity of the individual to learn is improved by the method of the invention. The type of learning may be discrimination learning, decision-making, delayed reinforcement learning, or reversal learning.

In any aspect of the invention, the individual may be administered a clinically safe level of acoustic energy at least once, twice, three times, four times, five times, six times or more. In a preferred aspect, the treatment is administered six times or more over a six week period.

In any aspect of the invention, the method may be conducted without the addition of an exogenous therapeutic agent.

An individual may be identified as having a neurodegenerative disease caused by or characterized by the pathological presence of one or more of the proteins selected from the group consisting of Amyloid beta, amyloid fragments, amyloid precursor protein, amyloid precursor protein fragments, tau and British peptide. Preferably the individual does not have imageable deposits of one or more proteins selected from the group consisting of Amyloid beta, amyloid fragments, amyloid precursor protein, amyloid precursor protein fragments, tau and British peptide. Further an individual may be identified as having a neurodegenerative disease if oligomers, aggregates or deposits of one or more pathogenic proteins, including those described herein, are biochemically or clinically detectable.

Typically, an improvement in cognitive function, for example memory or learning, is determined by any means as described herein including standardised neuropsychological testing.

In any aspect of the method of the invention, the method further comprises a step of identifying a region of the brain of the individual to be treated with acoustic energy.

Preferably the region of the brain is one known to be related to regulation of memory. Preferably the region is any one or more regions of the brain described herein.

In any aspect, the method of the invention further comprises determining a plurality of discrete application sites for application of acoustic energy to saturate or substantially saturate the region with acoustic energy.

In any aspect, the method further comprises determining a scanning path along which acoustic energy is to be applied to saturate or substantially saturate the region with acoustic energy. Preferably, the method further includes determining a plurality of discrete application sites for application of acoustic energy along the scanning path.

Typically, applying a clinically safe level of acoustic energy to the region includes providing acoustic energy continuously, or at application sites, along a scanning path.

In one embodiment, the scanning path is defined by a pre-determined pattern. The scanning path may be selected from the group consisting of linear, serpentine, a raster pattern, spiral and random.

Each application site may be spaced along the scanning path or each subsequent application site may overlap with the previous application site.

Applying a clinically safe level of acoustic energy to the region, includes applying acoustic energy at an application site such that a corresponding treatment volume is therapeutically affected by acoustic energy, and wherein saturating or substantially saturating the region with acoustic energy includes applying acoustic energy at a plurality of discrete application sites or one or more extended application sites such that the corresponding treatment volume(s) correspond substantially with the region.

The plurality of application sites may be selected such that treatment volumes of at least some sites overlap to form a group of treatment volumes that corresponds substantially with the region.

The plurality of application sites may be selected such that their corresponding treatment volumes overlap to form a contiguous treatment volume that corresponds substantially with the region.

The method can further include determining an order or application of acoustic energy at the plurality of application sites. The order or application of acoustic energy may be determined to apply a clinically safe level of acoustic energy. Typically this involves minimising any one or more of heating, brain swelling, red blood cell extravasation, haemorrhage or edema.

An order of application of acoustic energy to the plurality of application sites may be determined so that a minimum delay period is provided between an application of acoustic energy to application sites with adjacent or overlapping treatment volumes. Preferably, an order or application of acoustic energy does not include sequentially applying acoustic energy to application sites with adjacent or overlapping treatment volumes.

A region of the brain may the entire brain, hemisphere, forebrain or a region of the brain of the individual known to be associated with a condition involving the presence of proteins adopting pathogenic structures in an extracellular region. Such structures may be oligomers, aggregates and/or deposits. The region may be any one or more of the following cerebrum, cerebral hemisphere, telencephalon, forebrain, cortex, frontal lobe, prefrontal cortex, precentral gyrus, primary motor cortex, premotor cortex, temporal lobe, auditory cortex, inferior temporal cortex, superior temporal gyrus, fusiform gyrus, parahippocampal gyrus, entorhinal cortex, parietal lobe, somatosensory cortex, postcentral gyrus, occipital lobe, visual cortex, insular cortex, cingulate cortex, subcortical, hippocampus, dentate gyrus, cornu ammonis, amygdala, basal ganglia, striatum, caudate, putamen, nucleus accumbens, olfactory tubercle, globus pallidus, subthalamic nuclei, piriform cortex, olfactory bulb, fornix, mammillary bodies, basal forebrain, nucleus basalis Meynert, diencephalon, thalamus, hypothalamus, midbrain, tectum, tegmentum, substantia nigra, hindbrain, myelencephalon, medulla oblongata, metencephalon, pons, cerebellum, spinal cord, brain stem and cranial nerves.

Preferably, a clinically safe level of acoustic energy does not result in detectable heating, brain swelling, red blood cell extravasation, haemorrhage or edema.

Acoustic energy used in the invention may be ultrasound. Ultrasound may be focussed or unfocussed.

In any aspect of the present invention, the method does not comprise a step of administering microbubbles. Preferably, the method does not comprise a step of administering an agent that promotes cavitation. More preferably, the method does not comprise the step of administering an agent to promote an increase in permeability of the BBB.

In any aspect of the present invention, the aged individual does not contain microbubbles at the time the acoustic energy is applied. Preferably, the individual does not contain an agent that promotes cavitation at the time the acoustic energy is applied. More preferably, the individual does not contain an agent to promote an increase in permeability of the BBB at the time the acoustic energy is applied.

Alternatively, in any aspect of the present invention, the method further comprises a step of administering an agent to promote the increase in permeability of the blood-brain barrier. In a preferred form that agent promotes cavitation. An agent that promotes cavitation may be a microbubble agent as described herein. The microbubble may be provided to the subject by continuous infusion or a single bolus. The infusion may occur sequentially to, or following the start of, or simultaneously with, the application of the acoustic energy.

Alternatively, in any aspect of the present invention, the aged individual does contain an agent to promote the increase in permeability of the blood-brain barrier. In a preferred form that agent promotes cavitation. An agent that promotes cavitation may be a microbubble agent as described herein.

Any aspect of the present invention, the method of the invention described herein may further comprise the step of determining that the permeability of the blood-brain barrier has increased.

Any aspect of the present invention, the method of the invention described herein may further comprise the step of positioning at least one ultrasound emitter at an anatomical location proximate to a region of the brain known to be involved in cognitive function, preferably learning, memory formation and/or memory retrieval.

The acoustic energy may be applied in a method of the invention at a pressure greater than 0.4 MPa. Typically this pressure is used when application of the acoustic energy is outside the skull, i.e. transcranially. Otherwise, the acoustic energy may be applied with a mechanical index of between 0.1 and 2.

In any method of the invention, the step of applying the acoustic energy may be repeated.

Typically, the application of the acoustic energy in a method of the invention is not image-guided.

Another embodiment of the invention is directed to an apparatus for, or when used for, any of the methods described herein, comprising an ultrasound emitting device consisting of an ultrasound transducer with appropriate signal generation and amplification, and a fluid coupler for transmitting the ultrasonic output and a microbubble agent.

In a further aspect, the present invention provides an apparatus configured to perform any one or more of the methods described herein. The apparatus may comprise any one or more of the following: an acoustic energy emitter configured to emit acoustic energy for delivery to a region of the brain of the subject, a microbubble delivery device configured to deliver microbubbles to a region of the brain of the subject for disrupting the blood-brain barrier, and a controller that may control any one or more of the acoustic energy emitter and the microbubble delivery device. The apparatus may be used in conjunction with an imaging device, such as an MRI device, a positron emission tomography (PET) device, a computerized tomography (CT) or computerized axial tomography (CAT) device, or an ultrasound device. The apparatus may also be used in conjunction with an imaging contrast agent delivery device configured to deliver an imaging contrast agent to a region of the brain of the subject to aid in imaging of the brain by the imaging device. The imaging device and the imaging contrast agent may be controlled by the controller.

In another aspect, the present invention provides for a non-volatile machine readable medium, comprising instructions for configuring any apparatus described herein to perform any one or more of the methods described herein. In an aspect, the method is for improving or restoring cognitive function in an aged individual that does not have a neurodegenerative disease characterized by the presence of a pathological protein, wherein the method comprises or consists of:
applying a clinically safe level of acoustic energy to sites within a region of the brain, thereby saturating or substantially saturating the region with acoustic energy,
thereby improving or restoring cognitive function in the aged individual.

In another aspect, the present invention provides an apparatus for improving cognitive function in an aged individual as described herein (for example, who that does not have a neurodegenerative disease characterized by the presence of a pathological protein), the apparatus comprising an acoustic energy emitter controlled by a controller, the controller being adapted to cause the acoustic emitter to apply a clinically safe level of acoustic energy to a region of the brain, thereby saturating or substantially saturating the region with acoustic energy and wherein the region of the brain comprises an entire brain, a hemisphere, a forebrain, at least 25% by volume of the brain, or a region of the brain associated with cognitive impairment, and wherein the controller is adapted to control the acoustic emitter to saturate or substantially saturate the region with acoustic energy.

The controller and emitter may be adapted to apply the acoustic energy so as to achieve, in use, an increase in the permeability of the blood brain barrier of the brain to thereby improve cognitive function in the aged individual without requiring a therapeutic agent.

The controller may be adapted to control the acoustic emitter to saturate or substantially saturate the region with acoustic energy. Typically, the controller is adapted to do so without MRI guidance information being provided to the controller.

The controller may be adapted to cause the acoustic emitter to saturate an entire brain, a hemisphere, at least 25% by volume of the brain, or the forebrain with acoustic energy. Typically, the controller determines a plurality of discrete application sites for application of acoustic energy to saturate or substantially saturate the region with acoustic energy and causes the acoustic emitter to saturate the application sites with acoustic energy. Further, the controller may control the acoustic emitter to move along a scanning path along which acoustic energy is, in use, applied to saturate or substantially saturate the region with acoustic energy, preferably the apparatus including a motorised emitter positioning system controlled by the controller. The controller may cause the emitter, in use, to apply acoustic energy at a plurality of discrete application sites along the scanning path.

The controller may cause the emitter, in use, to apply a clinically safe level of acoustic energy to the region by providing acoustic energy continuously, or at application sites, along a scanning path defined by a pre-determined pattern, optionally or preferably the scanning path being selected from the group consisting of linear, serpentine, a raster pattern, spiral and random, and optionally wherein each application site is spaced along the scanning path.

The controller may be adapted to cause the emitter to emit and/or move such that each subsequent application site overlaps with the previous application site. Further, the controller may be adapted to achieve saturating or substantially saturating the region with acoustic energy causing the emitter to apply acoustic energy to a plurality of discrete application sites or one or more extended application sites such that the corresponding treatment volume(s) correspond substantially with the region, and optionally such that the plurality of application sites are selected such that treatment volumes of at least some sites overlap to form a group of treatment volumes that corresponds substantially with the region, and optionally wherein the plurality of application sites are selected such that their corresponding treatment volumes overlap to form a contiguous treatment volume that corresponds substantially with the region.

Typically, the controller determines an order or application of acoustic energy at the plurality of application sites. The order or application of acoustic energy to the plurality of application sites can be determined so that a minimum delay period is provided between of the emitter applying acoustic energy to application sites with adjacent or overlapping treatment volumes, and optionally wherein the controller controls the order or application of acoustic energy to the plurality of application sites so as not to sequentially apply acoustic energy to application sites with adjacent or overlapping treatment volumes.

The controller and emitter may be adapted to apply the acoustic energy so as to achieve, in use, an increase in the permeability of the blood-brain barrier of the brain, optionally by applying ultrasound with a mechanical index of between 0.1 and 2.

The controller may be adapted, in use, to control the emitter to apply ultrasound with a duty cycle of about 0.1% to about 50%, about 1% to about 20%, about 1% to about 10%, or about 1% to about 5%.

The controller may be adapted to cause the emitter to apply ultrasound with a pulse length of between about 1 to about 100 milliseconds, preferably about 1 to about 20 milliseconds, and/or with burst mode or pulse repetition frequencies of between about 0.1 to 10 Hz, 10 Hz to 100 kHz, 10 Hz to 1 kHz, 10 Hz to 500 Hz or 10 Hz to 100 Hz. Preferably, the burst mode or pulse repetition frequency is about 2 Hz.

The controller may be adapted to cause the emitter to apply ultrasound with a focal spot size of about a 1 mm to 2 cm, preferably 1 mm to 1.5 cm, preferably 1 mm to 1 cm, or preferably 1 mm to 0.5 cm axial width, and/or with a length of a focal spot of about 1 cm to about 15 cm, preferably 1 cm to 10 cm, preferably 1 cm to 5 cm.

An apparatus of the invention may further comprise a focused ultrasound system adapted to apply the acoustic energy, and optionally wherein the acoustic energy is applied transcranially at a pressure greater than 0.4 MPa, for example 0.7 MPa.

An apparatus of the invention may further comprise a blood-brain barrier increasing agent administrator adapted to administer an agent that, in use, promotes an increase in blood-brain barrier permeability, and wherein the controller is adapted to control the agent administrator, in use, and preferably wherein the agent comprises a cavitation promotion such as a microbubble agent. The agent administrator may comprise a microbubble infusion system or bolus introducer optionally controlled by the controller so as, in use, to administer the microbubbles sequentially to, or following the start of, or simultaneously with, the application of the acoustic energy by the emitter.

In any apparatus of the invention, the emitter comprises an ultrasonic transducer with ultrasonic signal generation and application means, and a fluid coupler for transmitting the ultrasonic output to the brain, and a microbubble agent administrator.

An apparatus of the invention may further comprise a supply of microbubble agent, and preferably an intravenous microbubble delivery means.

An apparatus of the invention may further comprise an imager such as an MRI imager, PET imager, CT or CAT imager adapted to be used to determine if there has been an increase in permeability of the blood-brain barrier or increase in temperature.

An apparatus of the invention may further comprise an image contrast agent administrator adapted, in use, to apply an image contrast agent to the brain to improve, in use, the imaging of the brain in order to determine the blood-brain barrier permeability.

The present invention also provides an injectable microbubble agent for use in improving cognitive function in an aged individual as described herein. Preferably, the microbubble agent is for, or when used for, any of the methods described herein.

Preferably, the microbubble agent has a lipid or polymer shell, and a gas stabilised core. Typically, the microbubble agent has a diameter of less than 10 μm.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Scanning ultrasound (SUS) treatment without microbubbles significantly improves spatial cognitive ability in aged mice. Following a six week treatment (SUS+microbubbles, SUS only, microbubbles only=sham), the aged mice were tested using the hippocampal-dependent Active Place Avoidance (APA) task, a test of spatial learning. The SUS only animals were able to avoid the aversive shock zone more effectively compared to Sham animals (A). The SUS only animals also improved their ability in terms of receiving less shocks received from the start to the finish of the test (B). This is clearly shown by less shocks received in the final five minutes for SUS only animals compared to the sham treatment group (C) (one-way ANOVA with Bonferroni post hoc test).

FIG. 2: LTP can be induced in aged animals following SUS treatment. (A) Following theta-burst stimulation (TBS) in vitro (indicated by a black arrow), robust LTP was observed in both the SUS and SUS without microbubbles groups, whilst LTP could not induced in the sham group (SUS and SUS without microbubbles: n=6-8 slices from 4 mice; sham: n=8 slices from 5 mice, mean±SEM). (B) Histogram representing the average of the last 10 min of LTP (mean±SEM) reveals a significant increase in LTP magnitude for both SUS treatments compared to the sham treatment (one-way ANOVA with Bonferroni post hoc test).

FIG. 3: SUS treatment increases synaptic activity and rescues LTP in the dentate gyrus of 22 month-old mice to a level of 18 month-old mice. (A). Left panel. Representative example of an input/output (I/O) curve for both naive 18 and 22 month-old, and SUS+microbubbles (22 month-old) treated mice (Of note: The curves for SUS with and without microbubbles overlap). Average of the I/O for both group (mean±SEM). SUS treatment increases synaptic transmission in the SUS-treated group. (B). Following a theta-burst stimulation (TBS) in vitro (indicated by a black arrow) no LTP has been observed in naive 22 month-old mice. On the opposite hand, LTP is fully rescued in SUS (22 month-old) treated mice to a level equivalent of naïve 18 month-old mice. Inset: representative trace before TBS (1) and the last ten minutes of recording (2; average of 20 traces). Right panel. Histogram representing the average of the last 10 minutes of LTP.

FIG. 4: SUS treatment increases neurogenesis. A. Numbers of doublecortin (Dcx)-positive cells were quantified per brain section in mice of sham, SUS treatment with microbubbles and SUS treatment alone groups.

### DETAILED DESCRIPTION OF THE EMBODIMENTS

Reference will now be made in detail to certain embodiments of the invention.

While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text or drawings. All of these different combinations constitute various alternative aspects of the invention. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All of the patents and publications referred to herein are incorporated by reference in their entirety. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

Throughout this specification, unless specifically stated otherwise or the context requires otherwise, reference to a single step, composition of matter, group of steps or group of compositions of matter shall be taken to encompass one and a plurality (i.e.

one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

One skilled in the art will recognize many methods and materials similar or equivalent to those described herein, which could be used in the practice of the present invention. The present invention is in no way limited to the methods and materials described.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

The present invention is based on the work performed by the inventors to determine whether the physiological ageing process of the brain (as distinct from pathological forms of ageing) can be halted by using acoustic energy, preferably in the form of ultrasound. The inventors used 22 month-old mice which is close to the end of the life-span of a mouse and represents physiologically aged humans (about 73-75 years old). Cognition in mice can be assessed in various ways including electrophysiologically for example by field recordings and behaviourally for example by performing an active place avoidance (APA) test. Importantly, the capacity to form memories is linked to the possibility of neurons in memory-forming brain areas such as the hippocampus to induce long-term potentiation (LTP) which is operationally defined as a long-lasting increase in synaptic efficacy following high-frequency stimulation of afferent fibers.

At 22 months of age, LTP cannot be induced in old C57BL/6 wild-type mice. The inventors surprising found that ultrasound treatments either with or without microbubbles fully restored the capacity of the aged mice to induce LTP. The inventors also surprisingly found in the APA paradigm, that the ultrasound only group without any microbubbles administered, significantly improved the performance in the APA test. Together this presents acoustic energy application as a cognition enhancement tool in physiologically aged humans.

Embodiments of the present invention have various advantages. The cognitive benefits, both the electrophysiological correlate of memory, and learning and memory retrieval, are observed in aged individuals, including very aged individuals. The benefits are observed in individuals with little or no capacity to induce long-term potentiation. The acuteness of the cognitive benefits in aged individuals, both the electrophysiological correlate of memory, and learning and memory retrieval, occurs almost immediately after application of acoustic energy. Further, the restoration of cognitive benefits in aged individuals, particularly the capacity to form memories, occurs to levels observed in young individuals. The beneficial effects of acoustic energy in the context of the present invention are not dependent on the presence of microbubbles and therefore microbubble mediated opening of the blood brain barrier (BBB) is not required.

In any aspect of the present invention, the aged individual is at least 40, 45, 50, 55, 60, 65, 70, 75, 80, 85, 90 or 95 years old. Preferably, the aged individual is at least 50, 55, 60, 65, 70, 75, 80, 85, 90 or 95 years old. Even more preferably, the aged individual is at least 60, 65, 70, 75, 80, 85, 90 or 95 years old.

By a “young” or “young individual” it is meant an individual that is of chronological age of 40 years old or younger, e.g., 35 years old or younger, including 30 years old or younger, e.g., 25 years old or younger or 22 years old or younger. As such, “young” and “young individual” may refer to a subject that is between the ages of 0 and 40, e.g., 0, 1, 5, 10, 15, 20, 25, 30, 35, or 40 years old.

As used herein, an individual that “does not have a neurodegenerative disease characterized by the presence of a pathological protein” means that the individual does not have any biochemically and/or clinically detectable neurodegenerative disease characterized by presence of a pathological protein. Preferably, the aged individual does not have any biochemically and/or clinically detectable neurodegenerative disease. Therefore, in any aspect or embodiment of the invention described herein, the aged individual does not have any biochemically and/or clinically detectable neurodegenerative disease. The neurodegenerative diseases to which the invention can be applied are those where pathogenic protein is extracellular and/or intracellular and causes or contributes to the disease or a symptom thereof. The pathogenic protein may be in pathogenic form when in an altered structure such as an oligomer, an aggregate or a deposit. Alzheimer's disease, dementia with Lewy bodies, Parkinson's disease, frontotemporal lobar degeneration and British and Danish familial dementia are non-limiting examples of diseases associated with extracellular pathogenic protein. Alzheimer's disease is the most common example of these diseases in which oligomers or plaques composed of amyloid beta (Aβ) are formed in the brain. Other neurodegenerative diseases are caused by, or associated with, the pathological aggregation of one or more of the proteins: Amyloid beta (Aβ), amyloid fragments, amyloid precursor protein, amyloid precursor protein fragments or British peptide. In any aspect, the individual may not have a neurodegenerative disease associated with the presence of a pathological protein.

An individual may be identified as having a neurodegenerative disease caused by, or associated with, the pathological aggregation of one or more of the proteins selected from the group consisting of Amyloid beta, amyloid fragments, amyloid precursor protein, amyloid precursor protein fragments, tau and British peptide.

Preferably the individual does not have imageable deposits of one or more proteins selected from the group consisting of Amyloid beta, amyloid fragments, amyloid precursor protein, amyloid precursor protein fragments, tau and British peptide. Further an individual may be identified as having a neurodegenerative disease if oligomers, an aggregate or deposit of one or more pathogenic proteins, including those described herein, are biochemically or clinically detectable.

In one aspect, a neurodegenerative disease may be cancer. Therefore, in any aspect, the individual does not have cancer or a clinically or biochemically detectable tumour, preferably brain tumour. In another aspect, the individual may have cancer, or a clinically or biochemically detectable tumour, that is not associated with a neurodegenerative disease.

“Tauopathies” are a class of neurodegenerative disorders resulting from the pathological function of tau, primarily the pathological aggregation of tau into filaments such as paired helical filaments (PHF) and eventually into aggregates such as neurofibrillary tangles (NFT). A “tauopathy” one of the class of neurodegenerative disorders resulting from the pathological function of tau, primarily the pathological aggregation of tau into neurofibrillary tangles (NFT). Examples of tauopathies include Alzheimer's disease, Amyotrophic lateral sclerosis/parkinsonism—dementia complex, Argyrophilic grain dementia, Corticobasal degeneration, Creutzfeldt-Jakob disease, Dementia pugilistica, Diffuse neurofibrillary tangles with calcification, Down's syndrome, Frontotemporal dementia with parkinsonism linked to chromosome 17a, Gerstmann-Sträussler-Scheinker disease, Hallervorden-Spatz disease, Myotonic dystrophy, Niemann-Pick disease, type C, Non-Guamanian motor neuron disease with neurofibrillary tangles, Pick's disease, Postencephalitic parkinsonism, Prion protein cerebral amyloid angiopathy, Progressive subcortical gliosis, Progressive supranuclear palsy, Subacute sclerosing panencephalitis and Tangle only dementia. An individual for treatment by a method or apparatus of the invention is not one that is identified as having a tauopathy.

In an aspect of the invention, there is provided a method of increasing neurogenesis in an aged individual that does not have a neurodegenerative disease characterized by presence of a pathological protein. Neurogenesis will be understood to mean a process by which nervous system cells or neurons, are produced by neural stem cells.

Neurogenesis can be determined by any known means in the art including, but not limited to determining numbers of cells that are positive for (a) doublecortin (DCX)—a microtubule-associated protein expressed by neuronal precursor cells and immature neurons in embryonic and adult cortical structures, as well as (b) glial fibrillary acidic protein, (c) nestin, (d) Pax6, (e) NeuroD, (f) polysialylated neuronal cell adhesion molecule (PSA-NCAM), (g) TUC-4 (TOAD (Turned On After Division]/Ulip/CRMP)), (h) Tuj-1 (Neuron-specific class III beta-tubulin), and (i) calretinin.

An individual for treatment by a method or apparatus of the invention is not one that is identified as having early, intermediate or late stage disease and in the case of Alzheimer's disease is not an individual identified as having either diffuse Aβ oligomers or plaques.

At a clinical level, Alzheimer's disease may present a number of cognitive symptoms including mental decline, difficulty thinking and understanding, depression, hallucination, or paranoia, confusion in the evening hours, delusion, disorientation, forgetfulness, making things up, mental confusion, difficulty concentrating, inability to create new memories, inability to do simple maths, or inability to recognise common things. Behavioural symptoms may also be present and include aggression, agitation, difficulty with self-care, irritability, meaningless repetition of own words, personality changes, lack of restraint, or wandering and getting lost. Loss of loss of appetite or restlessness may also be present.

Thus, when a patient presents to a doctor with any of the above symptoms, some of the commonly used diagnostic tests include cognitive tests. Cognitive tests are used to measure and evaluate cognitive, or ‘thinking’, functions such as memory, concentration, visual-spatial awareness, problem solving, counting and language skills. Particular cognitive tests that may be used include the following Mini-Mental Status Examination (MMSE), Alzheimer's Disease Assessment Scale-Cognitive (ADAS-Cog), Neuropsychological Testing, Brain imaging techniques. Various brain-imaging techniques are sometimes used to show brain changes and to rule out other conditions such as tumour, infarcts (strokes—dead areas of brain tissue) and hydrocephalus (fluid on the brain); these include Computed tomography (CT or CAT) scan, Magnetic Resonance Imaging (MRI), and Positron Emission Tomography (PET) and Single-Photon Emission Computerized Tomography (SPECT).

“Cognitive function” or “cognitive status” refers to any one or more higher order intellectual brain process or brain state, respectively, involved in learning and memory including, but not limited to, attention, information acquisition, information processing, working memory, spatial memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behaviour, and expressing an interest in one's surroundings and self-care. As used herein, types of memory include any one or more of working memory, short-term memory, long-term memory, anterograde memory, retrograde memory, and memory retrieval.

“Cognitive impairment” or “CI” or an equivalent construct, such as “impaired cognitive function” or “cognitive decline”, refers to a deficit or reduction (e.g., by about 10%, 30%, 50%, 75%, 90% or 95%) in cognitive status or cognitive function, as defined above, compared to that same function in an age-matched control subject or more usually a population. As used herein, this generally refers to impairment not associated with, or caused by, the presence of a pathological protein, preferably the presence of an oligomer, aggregate and/or deposit of a pathological protein. Further, as used herein, cognitive impairment generally refers to impairment not associated with a biochemical or clinical diagnosis of a neurodegenerative disease.

“Mild Cognitive Impairment” or “MCI” refers to a condition characterized by isolated memory impairment accompanied by no other cognitive abnormality and relatively normal functional abilities. One set of criteria for a clinical characterization of

MCI specifies the following characteristics: (1) memory complaint (as reported by patient, informant, or physician), (2) normal activities of daily living (ADLs), (3) normal global cognitive function, (4) abnormal memory for age (defined as scoring more than 1.5 standard deviations below the mean for a given age), and (5) absence of indicators of dementia (as defined by DSM-IV guidelines). Diagnosis of MCI usually entails an objective assessment of cognitive impairment, which can be garnered through the use of well-established neuropsychological tests, including the Mini Mental State Examination (MMSE), the Cambridge Neuropsychological Test Automated Battery (CANTAB) and individual tests such as Rey Auditory Verbal Learning Test (AVLT), Logical Memory Subtest of the revised Wechsler Memory Scale (WMS-R) and the New York University (NYU) Paragraph Recall Test.

In humans, the level of cognitive impairment, and subsequent improvement after a method of the invention, may be measured by various neuropsychological tests, alone or in combination, including, but not limited to, the Alzheimer's Disease Assessment Scale-cognitive subscale (ADAS-cog); Global Deterioration Scale (GDS); the clinical global impression of change scale (CIBIC-plus scale); the Alzheimer's Disease Cooperative Study Activities of Daily Living Scale (ADCS-ADL); the Mini Mental State Exam (MMSE); the Neuropsychiatric Inventory (NPI); the Clinical Dementia Rating Scale (CDR); the Rey Auditory Verbal Learning Test (AVLT), Logical Memory Subtest of the revised Wechsler Memory Scale (WMS-R); the New York University (NYU) Paragraph Recall Test the Cambridge Neuropsychological Test Automated Battery (CANTAB) or the Sandoz Clinical Assessment-Geriatric (SCAG).

In non-human mammalian models, for example, a rat or non-human primate model, the level of cognitive function may be measured by methods including, but not limited to, using a maze in which subjects use spatial information (e.g., Morris water maze, Barnes circular maze, elevated radial arm maze, elevated plus maze, T-maze and others), recognition tests using odor and novel objects, conditioning tests (e.g., fear conditioning, discrimination tasks, active avoidance, illuminated open-field, two-compartment exploratory test, second and third order conditioning tasks), and tests of higher level executive function (e.g., serial reaction time tests, delayed match and non-match to sample, and stimulus-reward associations including choices involving delayed reinforcement).

In addition, the level of cognitive function may be measured in mammals, including humans, using neuroimaging techniques, e.g., Positron Emission Tomography (PET), magnetic resonance imaging (MRI), functional magnetic resonance imaging (fMRI), Single Photon Emission Computed Tomography (SPECT), or any other imaging technique that allows one to measure brain function. The level of cognitive function in aging can be tested by any of the above methods using aged mammals.

“Improving” cognitive function refers to affecting impaired cognitive function so that it more closely resembles the function of an aged-matched normal, unimpaired subject, and includes affecting states in which cognitive function is reduced, e.g., by about 10%, 30%, 50%, 15%, 90% or 95% as compared to a normal subject. Cognitive function may be promoted to any detectable degree, but preferably is promoted sufficiently to allow an impaired subject to carry out daily activities of normal life. One or more cognitive functions may be improved to such an extent that they resemble cognitive functions in young individuals. Improvement in accordance with this invention also includes the alleviation or amelioration of one or more manifestations of cognitive impairment. A beneficial alteration of a cognitive function by this invention includes, but is not limited to, a change in cognitive function sufficient to result in an improved score in a test of cognitive function; the improvement of cognitive function in a subject with impaired cognitive function so that it more closely resembles, or does resemble, the function of a control subject, preferably, e.g., a young subject or an aged unimpaired subject; or the improvement over an aged cognitively impaired subject or population.

Methods of the invention also relate to “preserving” cognitive function which refers to affecting impaired cognitive function such that it does not decline or does not fall below that observed in the subject upon first presentation or diagnosis. Specifically, preserving cognitive function may be the delay of onset or slowing of the progression of cognitive impairment.

“Restoring” cognitive function refers to affecting impaired cognitive function so that it resembles the function of an aged-matched normal, unimpaired subject, or so that it resembles the function of a younger subject. For example, restoring cognitive function may result in the subject having one or more cognitive functions that resembles a subject 5, 10, 15, 20, 25, 30, 35 or more years younger. A beneficial alteration of a cognitive function by this invention includes, but is not limited to, a change in cognitive function sufficient to resemble, the function of a control subject or population, preferably, e.g., a young subject or an aged unimpaired subject or population.

Acoustic energy, such as ultrasound, can be applied to the entire brain or a region of the brain. A region of the brain may be a hemisphere or forebrain. The region may be at least 25% by volume of the brain. The region of the brain may be one that is known to be associated with one or more cognitive domains, preferably learning, memory formation and/or memory retrieval. Preferably, the region of the brain is associated with spatial learning, for example the hippocampus.

Identifying a region of the brain to which acoustic energy is applied may include determining a volume of the brain on the basis of symptoms displayed by the individual, typically clinically observable or biochemically detectable symptoms, or determining a volume of the brain on the basis of a known association with a cognitive domain.

The focus of the acoustic energy source, typically an ultrasound transducer, may be moved in a pattern with space between the individual sites of application over a region of the brain as described herein or the entire brain. The focus may be moved by a motorised positioning system.

In a preferred form, the methods of the invention involve the application of focussed ultrasound to a plurality of locations in the brain. The focussed ultrasound may be applied at 2, 3, 4, 5, 6, 7, 8, 9, 10 or more locations in the brain or on each hemisphere.

Some embodiments of the invention involved increasing permeability of the blood-brain barrier. Increasing the permeability of the blood-brain barrier can be promoted by various agents. These agents are based on the principle that biologically inert and preformed microbubbles, with either a lipid or polymer shell, a stabilized gas core, and a diameter of less than 10 μm, can be systemically administered and subsequently exposed to noninvasively delivered focused ultrasound pulses. Microbubbles within the target volume thereby become “acoustically activated” by what is known as acoustic cavitation. In this process, the microbubbles expand and contract with the acoustic pressure rarefaction and compression over several cycles. This activity has been associated with a range of effects including the displacement of the vessel wall through dilation and contractions. It is believed that the mechanical interaction between ultrasound, microbubbles and the vasculature transiently opens tight junctions thereby increasing the permeability of the blood-brain barrier.

The microbubble agent can be any agent known in the art including lipid-type microspheres or protein-type microspheres or a combination thereof in an injectable suspension. For example, the agent can be selected from the group consisting of Octafluoropropane/Albumin (Optison), a perflutren lipid microsphere (Definity), Galactose-Palmitic Acid microbubble suspension (Levovist) Air/Albumin (Albunex and Quantison), Air/Palmitic acid (Levovist/SHU508A), Perfluoropropane/Phospholipids (MRX115, DMP115), Dodecafluoropentane/Surfactant (Echogen/QW3600),

Perfluorobutane/Albumin (Perfluorocarbon exposed sonicated dextrose albumin), Perfluorocarbon/Surfactant (QW7437), Perfluorohexane/Surfactant (Imagent/AF0150), Sulphur hexafluoride/Phospholipids (Sonovue/BR1), Perfluorobutane/Phospholipids (BR14), Air/Cyanoacrylate (Sonavist/SHU563A), and Perfluorocarbon/Surfactant (Sonazoid/NC100100).

The microbubble agent may be provided as a continuous infusion or as a single bolus dose. A continuous infusion of microbubble, preferably provided over the duration of the ultrasound application, would be preferred. Typically, the microbubble agent is delivered intravenously through the systemic circulation.

For methods of the invention that include the use of an agent such as a microbubble or other cavitation based promotion of blood-brain barrier permeability, the agent may be localized at, or near, or in a region that is targeted with the ultrasound such that the potential of unwanted damage from cavitation effects is minimised.

The applying step, for the delivery of ultrasound, may comprise the delivery of ultrasound from an ultrasound source through a fluid coupler applied directly to the head of the subject. In this application, the fluid coupler may be applied to only one side or aspect of the subject's head. The head may be an unmodified head or a head with a surgically created window in the skull—the fluid coupler being in contact with the window. The ultrasound may be generated by an unfocused ultrasound transducer or a phased array ultrasound transducer (i.e., focused ultrasound). Significantly, the phased array ultrasound transducer may be a diagnostic phased array. Diagnostic phased arrays are generally of lower power and are commonly available. The fluid coupler may comprise a contained volume of fluid (e.g., about 50 cc, about 100 cc, about 200 cc, about 400 cc, about 500 cc, about 600 cc or about 1 litre). The fluid may be, for example, water, ultrasonic gel, or a substance of comparable acoustic impedance. The fluid may be contained in a fluid cylinder with at least a flexible end portion that conforms to the subject's head. In other embodiments, the contained volume of fluid may be a flexible or elastic fluid container.

Increased permeability of the blood-brain barrier may be determined by any suitable imaging method. Preferably, the imaging method is MRI, an optical imaging method, positron emission tomography (PET), computerized tomography (CT) or computerized axial tomography (CAT) or ultrasound. If a level of acoustic energy is applied, the increased permeability of the blood-brain barrier could then be determined by any one of the methods described herein and an increased level of acoustic energy could be subsequently applied until the permeability of the blood-brain barrier had increased to a clinically relevant level.

Any ultrasound parameters that result in clinically safe application of acoustic energy are useful in the invention. Typically, the ultrasound parameters that are preferred as those that result in an increase the permeability of the blood-brain barrier , or activate microglia phagocytosis. Various ultrasound parameters can be manipulated to influence the permeability increase in the blood-brain barrier and these include pressure amplitude, ultrasound frequency, burst length, pulse repetition frequency, focal spot size and focal depth. Several parameters are now described that are useful in a method of the invention.

Focal spot size useful in a method of the invention includes about a 1 mm to 2 cm axial width. Typically, the focal spot size has an axial width of about 1 mm to 1.5cm, preferably 1 mm to 1 cm, even more preferably 1 mm to 0.5 cm. The length of the focal spot may be about 1 cm to as much as about 15 cm, preferably 1 cm to 10 cm, even ore preferably 1cm to 5cm. The focal size useful in a method of the invention is one that allows an increase in the permeability of the blood-brain barrier of the subject.

The focal depth of the ultrasound generally depends on the areas of the brain affected by the disease. Therefore, the maximum focal depth would be the measurement from the top of the brain to the base, or about 10 to about 20 cm. Focal depth could be altered by electronic focusing, preferably by using an annular array transducer.

Typically the ultrasound is applied in continuous wave, burst mode, or pulsed ultrasound. Preferably the ultrasound is applied in burst mode, or pulsed ultrasound.

Pulse length parameters that are useful in a method the invention include between about 1 to about 100 milliseconds, preferably the pulse length or burst length is about 1 to about 20 milliseconds. Exemplary burst mode repetition frequencies can be between about 0.1 to 10 Hz, 10 Hz to 100 kHz, 10 Hz to 1 kHz, 10 Hz to 500 Hz or 10 Hz to 100 Hz.

The duty cycle (% time the ultrasound is applied over the time) is given by the equation duty cycle=pulse length x pulse repetition frequency×100. Typically, the duty cycle is from about 0.1% to about 50%, about 1% to about 20%, about 1% to about 10%, or about 1% to about 5%.

In some embodiments, the ultrasound pressure useful in a method of the invention is the minimum required to increase the permeability of the blood-brain barrier.

The human skull attenuates the pressure waves of the ultrasound which also depends on the centre frequency of the transducer, with lower centre frequencies of the ultrasound transducer causing better propagation and less attenuation. A non-limiting example of ultrasound pressure is between 0.1 MPa to 2 MPa, preferably about 0.4 or 0.5 MPa. Typically this pressure is applied to the skull, i.e transcranially. The mechanical index characterises the relationship between peak negative pressure amplitude in situ and centre frequency with mechanical index=Pressure (MPa)/sqrt centre frequency (MHz) if this mechanical index was free from attenuation/measured from within the skull, the mechanical index would be between about 0.1 and about 2, preferably about 0.1 to 1 or 0.1 to 0.5.

A non-limiting example of a system that is able to open the blood-brain barrier is the TIPS system (Philips Research). It consists of a focused ultrasound transducer that generates a focused ultrasound beam with a centre frequency of 1-1.7 MHz focal depth of 80 mm, active outer diameter 80 mm, active inner diameter 33.5 mm which is driven by a programmable acoustic signal source within the console and attached to a precision motion assembly. An additional example of a system that is able to generate an ultrasound beam suitable for blood-brain barrier disruption is the ExAblate Neuro® (Insightec) system.

For any of the method or apparatus of the invention, the ultrasound transducer may have an output frequency of between 0.1 to 10 MHz, or 0.1 to 2 MHz. The ultrasound may be applied for a time between 10 milliseconds to 10 minutes. The ultrasound may be applied continuously or in a burst mode.

Image contrast agents, used in any methods of the invention, may be selected from the group consisting magnetic resonance contrast agents, x-ray contrast agents (and x-ray computed tomography), optical contrast agents, positron emission tomography (PET) contrast agents, single photon emission computer tomography (SPECT) contrast agents, or molecular imaging agents. For example, the imaging contrast agent may be selected from the group consisting of gadopentetate dimeglumine, Gadodiamide, Gadoteridol, gadobenate dimeglumine, gadoversetamide, iopromide, Iopamidol, Ioversol, or Iodixanol, and lobitridol.

The frequency of application of the ultrasound would generally depend on patient severity. The parameters of the ultrasound and the treatment repetition are such that there is an increase in permeability of the blood-brain barrier but preferably wherein there is no, or clinically acceptable levels of, damage to parenchymal cells such as endothelial or neuronal damage, red blood cell extravasation, haemorrhage, heating and/or brain swelling.

Any method of the invention may further include performing magnetic resonance imaging on a subject comprising the steps of (a) administering a magnetic resonance contrast agent to a subject through the blood-brain barrier using any of the methods of the invention and performing magnetic resonance imaging on said subject. In this context the use of magnetic resonance imaging is to confirm the increase in permeability of the blood-brain barrier and not to locate the presence of a pathogenic protein.

Another embodiment of the invention involves providing an imaging contrast agent to the whole brain including the steps of administering an imaging contrast agent into the bloodstream of said subject; and applying ultrasound to the brain of said subject to open the blood-brain barrier to allow the image contrast agent to cross the blood-brain barrier. The imaging contrast agent can be administered to the subject simultaneously or sequentially with the application of the ultrasound. In this embodiment the sequential administration of the contrast agent can be prior to or post application of the ultrasound. In a preferred embodiment, any of the agents described herein may be administered to the bloodstream between 1 to 4 hours, between 2 to 4 hours or between 3-4 hours after ultrasound treatment using one of the methods of the invention.

### EXAMPLES

### Experimental Procedures

Animal ethics. Female C57BI/6 mice were used in this study. Animal experimentation was approved by the Animal Ethics Committee of the University of Queensland (approval number QBI/412/14/NHMRC).

SUS equipment. An integrated focused ultrasound system was used (Therapy Imaging Probe System, TIPS, Philips Research). The system consisted of an annular array transducer with a natural focus of 80 mm, a radius of curvature of 80 mm, a spherical shell of 80 mm with a central opening of 31 mm diameter, a 3D positioning system, and a programmable motorized system to move the ultrasound focus in the x and y planes to cover the entire brain area. A coupler mounted to the transducer was filled with degassed water and placed on the head of the mouse with ultrasound gel for coupling, to ensure propagation of the ultrasound to the brain. The focal zone of the array was an ellipse of approximately 1.5 mm×1.5 mm×12 mm.

Production of microbubbles. Lipid-shelled microbubbles with an octafluoropropane core were manufactured and characterized in-house. A 1:5:2:1 mass ratio of PEG6000, distearoyl-phosphatidylcholine, distearoylphosphatidylethanolamine, and pluronic F68 was dissolved in a 0.9% solution of sodium chloride. The solution was added to glass HPLC vials and the air was removed and replaced with octafluoropropane gas to fill the headspace of the vial (Arcadophta). On the day of use, vials were heated to 37° C. and then shaken in a dental amalgamator for 40 s at 4,000 rpm. The concentration and size of the microbubbles was examined under a microscope and found to be 1-5×10⁷ microbubbles/ml with a size range of 1-10 μm, and a mean diameter of 4 μm.

SUS application. Mice were anesthetized with zoletil (20 mg/kg) and xylazine (10 mg/kg) and the hair on the head was shaved and depilated. Mice were injected retro-orbitally with 1 μl/g body weight of microbubble solution and then placed under the ultrasound transducer with the head immobilized (intravenous injections were also tested but proved less efficacious due to the small tail veins of the mice). Parameters for the ultrasound delivery were 0.7 MPa peak rarefactional pressure, 10 Hz pulse repetition frequency, 10% duty cycle, and a 6 s sonication time per spot. The motorized positioning system moved the focus of the transducer array in a grid with 1.5 mm between individual sites of sonication so that ultrasound was delivered sequentially to the entire brain. For naive/sham treatment, mice received all injections and were placed under the ultrasound transducer, but no ultrasound was emitted.

Electrophysiology. Mice were deeply anaesthetized with isoflurane, perfused transcardially with ice-cold cutting solution (in mM; 93 NMDG, 2.5 KCl, 1.2 NaH₂PO₄, 30 NaHCO₃, 20 HEPES, 25 glucose, 5 Sodium Ascorbate, 2 Thiourea, 3 Sodium Pyruvate, 10 MgSO₄, 0.5 CaCl₂, PH7.3 adjusted with HCl, osmolarity 300-310 mOsm/kg) and subsequently decapitated. The brain was rapidly removed and coronal brain slices (300 pm thick for patch-clamp and 400 μm for field recordings; Leica VT1000S vibratome) were prepared in ice-cold cutting solution. Following dissection, slices were placed to recover in oxygenated artificial cerebrospinal fluid (aCSF in mM; 118 NaCl, 25 NaHCO₃, 10 glucose, 2.5 KCl 2.5, 1.2 NaHPO₄, 1.3 MgCl₂, 2.5 CaCl₂) for 30 mins at 32° C. before being allowed to equilibrate at room temperature for at least an additional 30 mins. Slice were visualized using an upright microscope (Olympus BX5OWI, Japan) and Field potential were recorded using a Multiclamp 700B amplifier (molecular devices, USA). During recording, slices were perfused with heated aCSF (30±2° C.). Recording pipettes were prepared from borosilicate glass (GC150F, Harvard Apparatus, UK) and pulled to a tip resistance of 3-6 MΩ (Narishige PC-10) when filled with aCSF. Local electrical stimulation of the medial performant pathway was evoked using a theta glass pipette. Long-term potentiation was induced by theta burst protocol (10 trains at 5 Hz of 10 pulses at 100 Hz repeated 3 times, 20 s apart.). During baseline and after theta burst protocol field were evoked by a single pulse every 30 s.

Active place avoidance (APA) test. The APA task is a test of hippocampus-dependent spatial learning. The mice were tested in a rotating elevated arena (Bio-Signal group) that had a grid floor and a 32 cm high clear plastic circular fence enclosing a total diameter of grid of 77 cm. High-contrast visual cues were present on the walls of the testing room. The arena and floor was rotated at a speed of 1 rpm and a 500 ms, 60 Hz, 0.5 mA mild shock was delivered through the grid floor when the animal entered a 60 degree shock zone, and every 1,500 ms until the animal left the shock zone. The shock zone was maintained at a constant position in relation to the room. Recorded tracks were analyzed with Track Analysis software (Bio-Signal group). In all behavioural tests, examiners were blinded to treatment. Data was analyzed with a Two-Way ANOVA with day of testing as a within-subjects factor and simple effects of group tested with Bonferoni post-hoc test.

### Results

The inventors used 22 month-old mice which is close to the end of the life-span of a mouse and represents physiologically aged humans of about 73-75 years old. Cognition in mice can be assessed in various ways including electrophysiologically for example by field recordings and behaviourally for example by performing an active place avoidance (APA) test to assess spatial memory. Importantly, a read-out and correlate of neurons to form memories is linked to the possibility of neurons in memory-forming brain areas such as the hippocampus to induce long-term potentiation (LTP) which is operationally defined as a long-lasting increase in synaptic efficacy following high-frequency stimulation of afferent fibers. Other readouts for memory and learning are tests such as the Morris water maze paradigm; however, this test is stressful to mice, and aged animals tend to float during the task. Instead, the hippocampus-dependent active place avoidance (APA) paradigm was chosen, a test the inventors previously demonstrated to be suitable for assessing cognition in Alzheimer's mice.

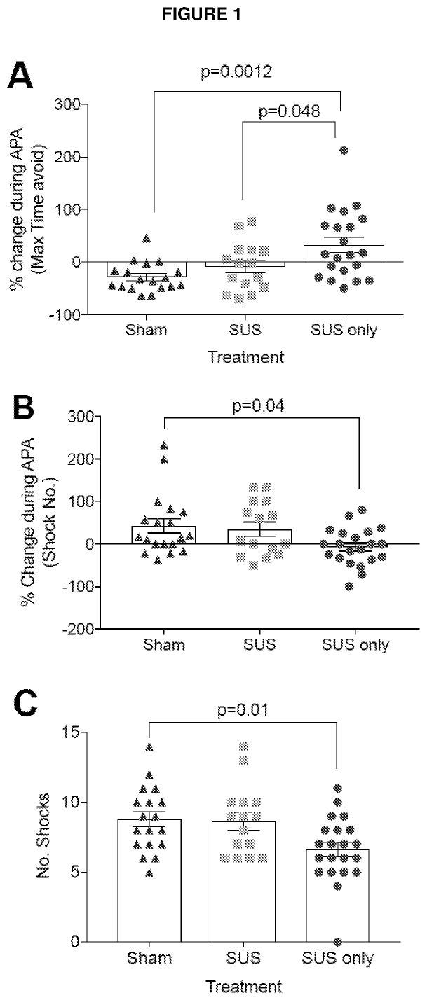

Mice aged 22 months were used in the study. Following a six week treatment (SUS+microbubbles, SUS only, microbubbles only=sham), the aged mice were tested using the hippocampal-dependent Active Place Avoidance (APA) task, a test of spatial learning. The SUS only animals were able to avoid the aversive shock zone more effectively compared to Sham animals (see FIG. 1A). The SUS only animals also improved their ability in terms of receiving less shocks received from the start to the finish of the test (see FIG. 1B). This is clearly shown by less shocks received in the final five minutes for SUS only animals compared to the sham treatment group (see FIG. 1C) (one-way ANOVA with Bonferroni post hoc test). The results of this hippocampus-dependent spatial learning test are that ultrasound treatment without microbubbles significantly improves spatial cognitive ability in aged mice. Further, the testing of the animals in the APA task occurred immediately after application of the ultrasound, revealing that the effects of ultrasound treatment are acute and immediate.

These results support the present invention that cognitive function (e.g. learning) in physiologically aged individuals, including those of greater than 40 years of age and even greater than 65 years of age, can be improved by ultrasound treatment without the use of an agent that promotes cavitation. Further, these results indicate that opening the blood brain barrier by transient disruption is not required.

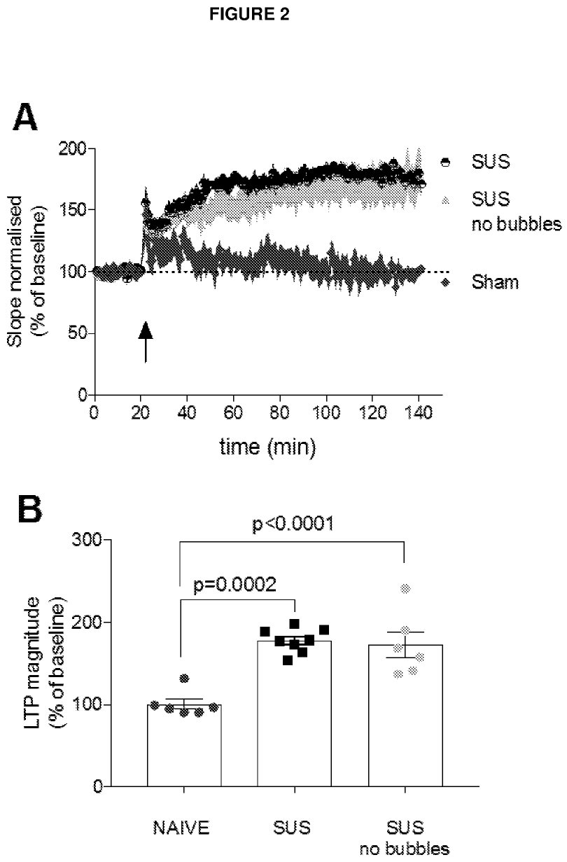

As shown in FIG. 2, LTP can be induced in aged animals following SUS treatment. Following theta-burst stimulation (TBS) in vitro (indicated by a black arrow), robust LTP was observed in both the SUS and SUS without microbubbles groups, whilst LTP could not induced in the sham group (SUS and SUS without microbubbles: n=6-8 slices from 4 mice; sham: n=8 slices from 5 mice, mean±SEM) (See FIG. 2A). The histogram in FIG. 2B representing the average of the last 10 min of LTP (mean±SEM) reveals a significant increase in LTP magnitude for both SUS treatments compared to the sham treatment (one-way ANOVA with Bonferroni post hoc test).

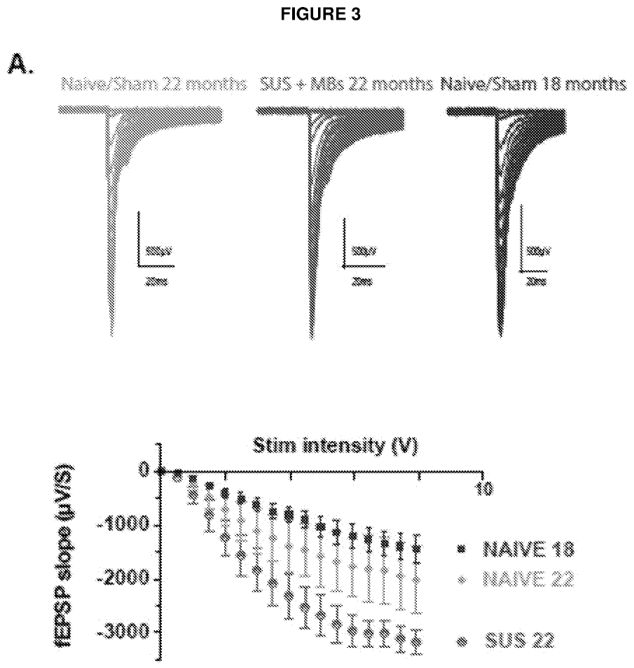

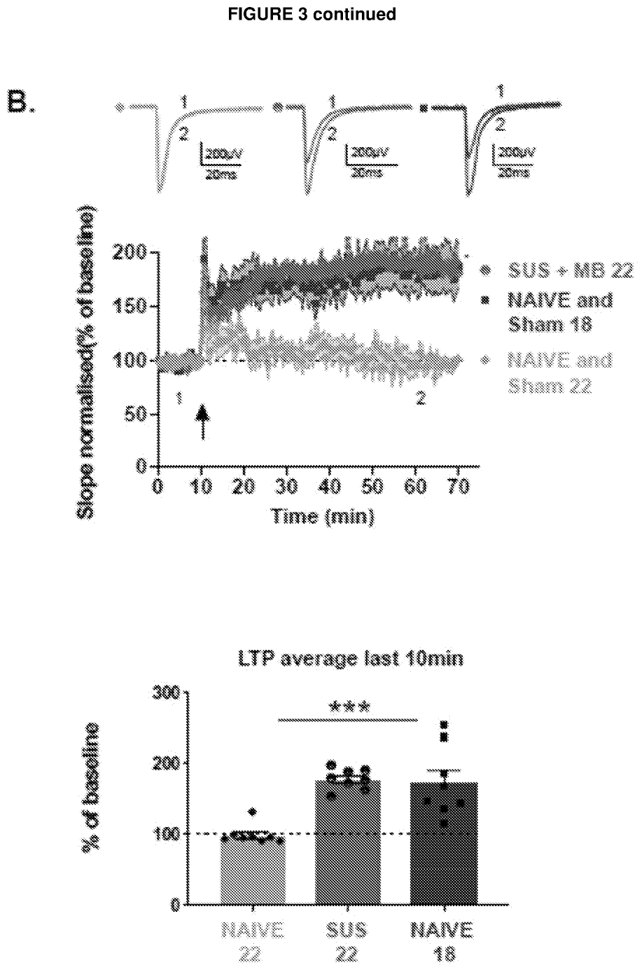

At 22 months of age, LTP cannot be induced in old C57BL/6 wild-type mice, but it can be induced in 18 month-old mice indicating that a significant decline occurs in mice between 18 and 22 months of age. The inventors surprisingly found that ultrasound treatments either with or without microbubbles fully restored the capacity of the aged mice to induce LTP (see FIG. 3B). Further, the inventors also found that ultrasound treatment of 22 month old mice increases synaptic transmission trending to a level greater than that observed in naïve 18 month old mice (see FIG. 3A). Similar to the APA test, the mice were analysed for induction of LTP and synaptic transmission without significant delay post ultrasound treatment.

The inventors also found that treatment with ultrasound increased the expression of pNR2B and NR2B (Glutamate [NMDA] receptor subunit epsilon-2) (data not shown). Analysis of brain sections revealed increases in the expression of signalling molecule ERK and pERK and decreases to the signalling molecule S6 and pS6 (data not shown).

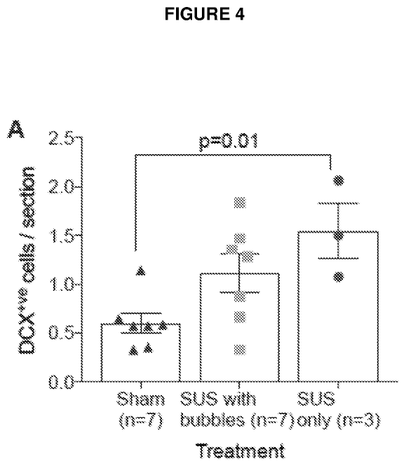

The inventors also assessed the effects of SUS treatment on neurogenesis by determining numbers of doublecortin (Dcx)-positive cells per section. It was found that that both treatments increased the numbers of Dcx-positive cells with a significant effect in response to SUS alone, demonstrating that ultrasound treatment can increase neurogenesis (FIG. 4A).

These results support the present invention that cognitive function (e.g. memory, specifically memory formation) in physiologically aged individuals, including those of greater than 40 years of age and even greater than 65 years of age, can be improved by ultrasound treatment, with or without the use of an agent that promotes cavitation. Further, these results indicate that opening the blood brain barrier by transient disruption is not necessarily required. The inventors found that statistically significant results were obtained according to the methods described herein.

It will be understood that the invention disclosed and defined in this specification extends to all alternative combinations of two or more of the individual features mentioned or evident from the text or drawings. All of these different combinations constitute various alternative aspects of the invention.

Without limitation, the invention may be summarised by the following items.

Item 1. A method of improving or restoring cognitive function in an aged individual that does not have a neurodegenerative disease characterized by the presence of a pathological protein, the method comprising:
applying a clinically safe level of acoustic energy to sites within a region of the brain, thereby saturating or substantially saturating the region with acoustic energy,
thereby improving or restoring cognitive function in the aged individual.

Item 2. A method according to item 1, wherein the cognitive function is in learning or memory.

Item 3. A method according to item 2, wherein the memory is short-term memory.

Item 4. A method according to item 2, wherein the memory is long-term memory.

Item 5. A method according to item 2, wherein the memory is memory retrieval.

Item 6. A method according to item 1, wherein the cognitive function is memory formation.

Item 7. A method according to item 2, wherein the learning is decision-making.

Item 8. A method according to any one of items 1 to 7, wherein the aged individual is at least 45 years old.

Item 9. A method according item 8, wherein the aged individual is at least 55 years old.

Item 10. A method according item 8, wherein the aged individual is at least 65 years old.

Item 11. A method according item 8, wherein the aged individual is at least 75 years old.

Item 12. A method according item 8, wherein the aged individual is at least 85 years old.

Item 13. A method according item 8, wherein the aged individual is at least 95 years old.

Item 14. A method according to any one of items 1 to 13, wherein the individual displays cognitive impairment of at least 10% compared to an age-matched control subject.

Item 15. A method according to any one of items 1 to 13, wherein the individual displays cognitive impairment of at least 30% compared to an age-matched control subject.

Item 16. A method according to any one of items 1 to 13, wherein the individual displays cognitive impairment of at least 50% compared to an age-matched control subject.

Item 17. A method according to any one of items 1 to 13, wherein the individual displays cognitive impairment of at least 70% compared to an age-matched control subject.

Item 18. A method according to any one of items 1 to 13, wherein the individual displays cognitive impairment of at least 90% compared to an age-matched control subject.

Item 19. A method according to any one of items 1 to 18, wherein the improvement in cognitive function results in the individual having a cognitive function similar to that of a young individual.

Item 20. A method according to item 19, wherein the young individual is 5 years younger than the aged individual.

Item 21. A method according to item 19, wherein the young individual is 10 years younger than the aged individual.

Item 22. A method according to item 19, wherein the young individual is 15 years younger than the aged individual.

Item 23. A method according to item 19, wherein the young individual is 20 years younger than the aged individual.

Item 24. A method according to item 19, wherein the young individual is 25 years younger than the aged individual.

Item 25. A method according to item 19, wherein the young individual is 30 years younger than the aged individual.

Item 26. A method according to item 19, wherein the young individual is 35 years younger than the aged individual.

Item 27. A method according to any one of items 1 to 26, wherein the method further comprises a step of identifying a region of the brain of the individual to be treated with acoustic energy.

Item 28. A method according to item 27, wherein the region of the brain is one known to be related to regulation of memory.

Item 29. A method according to any one of items 1 to 27, wherein the region of the brain is a hemisphere.

Item 30. A method according to any one of items 1 to 27, wherein acoustic energy is applied to the entire brain.

Item 31. A method according to any one of items 1 to 30, wherein the method of the invention further comprises determining a plurality of discrete application sites for application of acoustic energy to saturate or substantially saturate the region with acoustic energy.

Item 32. A method according to any one of items 1 to 31, wherein the method further comprises determining a scanning path along which acoustic energy is to be applied to saturate or substantially saturate the region with acoustic energy.

Item 33. A method according to item 32, wherein the method further comprises determining a plurality of discrete application sites for application of acoustic energy along the scanning path.

Item 34. A method according to any one of items 1 to 33, wherein a clinically safe level of acoustic energy does not result in detectable heating, brain swelling, red blood cell extravasation, haemorrhage or edema.

Item 35. A method according to any one of items 1 to 34, wherein the acoustic energy is ultrasound.

Item 36. A method according to item 35, wherein the ultrasound is focussed. Item 37. A method according to item 35, wherein the ultrasound is unfocussed. Item 38. A method according to any one of items 1 to 37, wherein the method does not comprise a step of administering microbubbles.

Item 39. A method according to any one of items 1 to 37, wherein the method does not comprise a step of administering an agent that promotes cavitation.

Item 40. A method according to any one of items 1 to 37, wherein the method does not comprise the step of administering an agent to promote an increase in permeability of the blood brain barrier.

Item 41. An apparatus adapted for, or when used for, any of the methods according to items 1 to 40, comprising an ultrasound emitting device consisting of an ultrasound transducer with appropriate signal generation and amplification, and a fluid coupler for transmitting the ultrasonic output and a microbubble agent.

Item 42. An apparatus configured to perform any one or more of the methods according to items 1 to 40, wherein the apparatus comprises any one or more of the following: an acoustic energy emitter configured to emit acoustic energy for delivery to a region of the brain of the subject, a microbubble delivery device configured to deliver microbubbles to a region of the brain of the subject for disrupting the blood-brain barrier, and a controller that may control any one or more of the acoustic energy emitter and the microbubble delivery device.

Item 43. An apparatus for improving cognitive function in an aged individual according to a method of any one of items 1 to 42, the apparatus comprising an acoustic energy emitter controlled by a controller, the controller being adapted to cause the acoustic emitter to apply a clinically safe level of acoustic energy to a region of the brain, thereby saturating or substantially saturating the region with acoustic energy and wherein the region of the brain comprises an entire brain, a hemisphere, a forebrain, at least 25% by volume of the brain, or a region of the brain associated with cognitive impairment, and wherein the controller is adapted to control the acoustic emitter to saturate or substantially saturate the region with acoustic energy.

Item 44. An apparatus according to item 42 or 43, wherein the controller and emitter are adapted to apply the acoustic energy so as to achieve, in use, an increase in the permeability of the blood brain barrier of the brain to thereby improve cognitive function in the aged individual without requiring a therapeutic agent.

Item 45. An apparatus according to item 42 or 43, wherein the controller is adapted to control the acoustic emitter to saturate or substantially saturate the region with acoustic energy, preferably the controller is adapted to do so without MRI guidance information being provided to the controller.

Item 46. An apparatus according to item 42 or 43, wherein the controller is adapted to cause the acoustic emitter to saturate an entire brain, a hemisphere, at least 25% by volume of the brain, or the forebrain with acoustic energy.

Item 47. An apparatus according to item 46, wherein the controller determines a plurality of discrete application sites for application of acoustic energy to saturate or substantially saturate the region with acoustic energy and causes the acoustic emitter to saturate the application sites with acoustic energy.

Item 48. An apparatus according to item 46 or 47, wherein the controller controls the acoustic emitter to move along a scanning path along which acoustic energy is, in use, applied to saturate or substantially saturate the region with acoustic energy, preferably the apparatus including a motorised emitter positioning system controlled by the controller.

Item 49. An apparatus according to item 48, wherein the controller causes the emitter, in use, to apply acoustic energy at a plurality of discrete application sites along the scanning path.

Item 50. An apparatus according to item 42 or 43, wherein the controller causes the emitter, in use, to apply a clinically safe level of acoustic energy to the region by providing acoustic energy continuously, or at application sites, along a scanning path defined by a pre-determined pattern, optionally or preferably the scanning path being selected from the group consisting of linear, serpentine, a raster pattern, spiral and random, and optionally wherein each application site is spaced along the scanning path.

Item 51. An apparatus according to item 42 or 43, wherein the controller is adapted to cause the emitter to emit and/or move such that each subsequent application site overlaps with the previous application site.

Item 52. An apparatus according to item 51, wherein the controller is adapted to achieve saturating or substantially saturating the region with acoustic energy causing the emitter to apply acoustic energy to a plurality of discrete application sites or one or more extended application sites such that the corresponding treatment volume(s) correspond substantially with the region, and optionally such that the plurality of application sites are selected such that treatment volumes of at least some sites overlap to form a group of treatment volumes that corresponds substantially with the region, and optionally wherein the plurality of application sites are selected such that their corresponding treatment volumes overlap to form a contiguous treatment volume that corresponds substantially with the region.

Item 53. An apparatus according to item 52, wherein the controller determines an order or application of acoustic energy at the plurality of application sites.

Item 54. An apparatus according to item 53, wherein the order or application of acoustic energy to the plurality of application sites can be determined so that a minimum delay period is provided between of the emitter applying acoustic energy to application sites with adjacent or overlapping treatment volumes, and optionally wherein the controller controls the order or application of acoustic energy to the plurality of application sites so as not to sequentially apply acoustic energy to application sites with adjacent or overlapping treatment volumes.

Item 55. An apparatus according to item 42 or 43, wherein the controller and emitter is adapted to apply the acoustic energy so as to achieve, in use, an increase in the permeability of the blood-brain barrier of the brain, optionally by applying ultrasound with a mechanical index of between 0.1 and 2.

Item 56. An apparatus according to item 42 or 43, wherein the controller is adapted, in use, to control the emitter to apply ultrasound with a duty cycle of about 0.1% to about 50%, about 1% to about 20%, about 1% to about 10%, or about 1% to about 5%.

Item 57. An apparatus according to item 42 or 43, wherein the controller is adapted to cause the emitter to apply ultrasound with a pulse length of between about 1 to about 100 milliseconds, preferably about 1 to about 20 milliseconds, and/or with burst mode repetition frequencies of between about 0.1 to 1 0Hz, 10 Hz to 100 kHz, 10 Hz to 1 kHz, 10 Hz to 500 Hz or 10 Hz to 100 Hz, preferably 2 Hz.

Item 58. An apparatus according to item 42 or 43, wherein the controller is adapted to cause the emitter to apply ultrasound with a focal spot size of about a 1 mm to 2 cm, preferably 1 mm to 1.5 cm, preferably 1 mm to 1 cm, or preferably 1 mm to 0.5 cm axial width, and/or with a length of a focal spot of about 1 cm to about 15 cm, preferably 1 cm to 10 cm, preferably 1 cm to 5 cm.

Item 59. An apparatus according to item 42 or 43, further comprising a focused ultrasound system adapted to apply the acoustic energy, and optionally wherein the acoustic energy is applied transcranially at a pressure greater than 0.4 MPa.

Item 60. An apparatus according to any one of item 42 to 43, wherein the emitter comprises an ultrasonic transducer with ultrasonic signal generation and application means, and a fluid coupler for transmitting the ultrasonic output to the brain, and a microbubble agent administrator.

Item 61. An apparatus according to items 42 or 43, further comprising a supply of microbubble agent, preferably an intravenous microbubble delivery means.

Item 62. An apparatus according to any one of items 42 to 60, further comprising an imager such as an MRI imager, PET imager, CT or CAT imager adapted to be used to determine if there has been an increase in permeability of the blood-brain barrier or increase in temperature.

Item 63. An apparatus according to any one of items 42 to 61, further comprising an image contrast agent administrator adapted, in use, to apply an image contrast agent to the brain to improve, in use, the imaging of the brain in order to determine the blood-brain barrier permeability.

Item 64. An injectable microbubble agent for use in, or when used in, improving or restoring cognitive function in an aged individual according to any one of items 1 to 40.

Item 65. An injectable microbubble agent according to item 64, wherein the microbubble agent has a lipid or polymer shell, and a gas stabilised core.

Item 66. An injectable microbubble agent according to item 64 or 65, wherein the microbubble agent has a diameter of less than 10 μm.

1. A method of improving or restoring cognitive function in an aged individual that does not have a neurodegenerative disease characterized by the presence of a pathological protein, the method comprising:
applying a clinically safe level of acoustic energy to sites within a region of the brain, thereby saturating or substantially saturating the region with acoustic energy,
thereby improving or restoring cognitive function in the aged individual.

2. A method according to claim 1, wherein the cognitive function is learning or memory.

3. A method according to claim 2, wherein the memory is short-term memory.

4. A method according to claim 2, wherein the memory is long-term memory.

5. A method according to claim 2, wherein the learning is decision-making.

6. A method according to any one of claims 1 to 5, wherein the aged individual is at least 45 years old.

7. A method according claim 6, wherein the aged individual is at least 65 years old.

8. A method according claim 6, wherein the aged individual is at least 75 years old.

9. A method according to any one of claims 1 to 8, wherein the individual displays cognitive impairment of at least 10% compared to an age-matched control subject.

10. A method according to claim 9, wherein the individual displays cognitive impairment of at least 50% compared to an age-matched control subject.

11. A method according to any one of claims 1 to 10, wherein the improvement in cognitive function results in the individual having a cognitive function similar to that of a young individual.

12. A method according to claim 11, wherein the young individual is 10 years younger than the aged individual.

13. A method according to claim 12, wherein the young individual is 20 years younger than the aged individual.

14. A method according to any one of claims 1 to 13, wherein the method of the invention further comprises determining a plurality of discrete application sites for application of acoustic energy to saturate or substantially saturate the region with acoustic energy.

15. A method according to any one of claims 1 to 14, wherein the acoustic energy is ultrasound.

16. A method according to claim 15, wherein the ultrasound is focussed.

17. A method according to claim 15, wherein the ultrasound is unfocussed.

18. A method according to any one of claims 1 to 17, wherein the method does not comprise a step of administering microbubbles.

19. A method according to any one of claims 1 to 17, wherein the method does not comprise the step of administering an agent to promote an increase in permeability of the blood brain barrier.

20. An apparatus adapted for, or when used for, any of the methods according to claims 1 to 19, comprising an ultrasound emitting device consisting of an ultrasound transducer with appropriate signal generation and amplification, and a fluid coupler for transmitting the ultrasonic output and a microbubble agent.

21. An apparatus for improving cognitive function in an aged individual according to a method of any one of claims 1 to 19, the apparatus comprising an acoustic energy emitter controlled by a controller, the controller being adapted to cause the acoustic emitter to apply a clinically safe level of acoustic energy to a region of the brain, thereby saturating or substantially saturating the region with acoustic energy and wherein the region of the brain comprises an entire brain, a hemisphere, a forebrain, at least 25% by volume of the brain, or a region of the brain associated with cognitive impairment, and wherein the controller is adapted to control the acoustic emitter to saturate or substantially saturate the region with acoustic energy.

22. An apparatus according to claim 21, wherein the controller determines a plurality of discrete application sites for application of acoustic energy to saturate or substantially saturate the region with acoustic energy and causes the acoustic emitter to saturate the application sites with acoustic energy.

23. An apparatus according to claim 21, wherein the controller is adapted to cause the emitter to apply ultrasound with a pulse length of between about 1 to about 100 milliseconds, preferably about 1 to about 20 milliseconds, and/or with burst mode repetition frequencies of between about 0.1 to 10 Hz, 10 Hz to 100 kHz, 10 Hz to 1 kHz, 10 Hz to 500 Hz or 10 Hz to 100 Hz, preferably 2 Hz.

24. An apparatus according to claim 21, further comprising a supply of microbubble agent, preferably an intravenous microbubble delivery means.

25. An injectable microbubble agent for use in, or when used in, improving or restoring cognitive function in an aged individual according to any one of claims 1 to 19.
